(19) United States
(12) Patent Application Publication
(10) Pub. No.: US 2026/0159466 A1
(43) Pub. Date: Jun. 11, 2026

(54) STILBENE DERIVATIVE, PREPARATION METHOD THEREOF AND APPLICATION THEREOF IN PREPARATION OF DRUGS FOR TREATING PSORIASIS OR ULCERATIVE COLITIS

(71) Applicant: Huazhong University of Science and Technology, Wuhan (CN)

(72) Inventors: Yonghui Zhang, Wuhan (CN); Weiguang Sun, Wuhan (CN); Lianghu Gu, Wuhan (CN); Yongqi Li, Wuhan (CN); Yunpeng Zhao, Wuhan (CN)

(21) Appl. No.: 19/183,802

(22) Filed: Apr. 19, 2025

Related U.S. Application Data
(63) Continuation of application No. PCT/CN2025/082349, filed on Mar. 13, 2025.

(30) Foreign Application Priority Data
Dec. 9, 2024 (CN) 2024117981291

(51) Int. Cl. C07C 39/21; A61K 31/05; A61K 31/085; C07C 41/26; C07C 41/30; C07C 43/23; C07C 67/00; C07C 67/10; C07C 67/24

(52) U.S. Cl. CPC C07C 39/21; A61K 31/05; A61K 31/085; C07C 41/26; C07C 41/30; C07C 43/23; C07C 67/00; C07C 67/10; C07C 67/24

(57) ABSTRACT

A stilbene derivative includes a compound II-2 and a compound II-23, and structural formulas of the compound II-2 and the compound II-23 are respectively as follows:

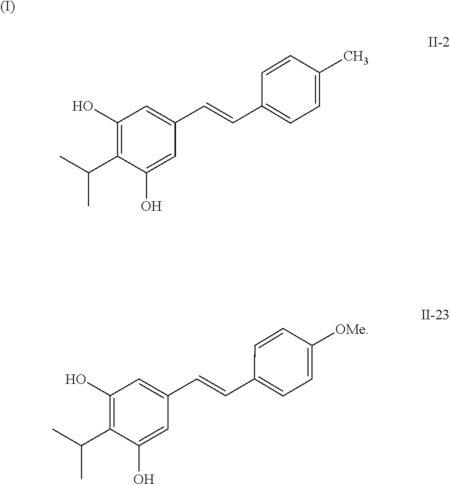

The compound II-2 and the compound II-23 can target activation of aryl hydrocarbon receptor (AHR) related pathways, promoting nuclear translocation of AHR. At cellular levels, the compound II-2 and the compound II-23 can reduce secretion of related cytokines, significantly improving skin symptoms and psoriasis area and severity index (PASI) scores of mice in an imiquimod-induced psoriasis mouse model. Compared to lead compound tapinarof, the compound II-2 and the compound II-23 exhibit superior anti-psoriasis activity and lower toxicity. In addition, the compound II-23 can effectively treat ulcerative colitis in mice, reducing inflammatory responses and improving colitis symptoms. The compound II-2 and the compound II-23 can be used to prepare drugs for treating psoriasis and ulcerative colitis.

### CROSS-REFERENCE TO RELATED APPLICATION

This application is a continuation of International Patent Application No. PCT/CN2025/082349, filed Mar. 13, 2025, which claims the priority of Chinese Patent Application No. 202411798129.1, filed Dec. 9, 2024, both of which are herein incorporated by reference in their entirety.

### TECHNICAL FIELD

The disclosure relates to field of biomedical technologies, and more particularly to a stilbene derivative, a preparation method thereof and an application thereof in preparation of drugs for treating psoriasis or ulcerative colitis.

### BACKGROUND

Psoriasis is a chronic, systemic, inflammatory skin disease associated with immune inflammation, characterized by red macules and plaques with silvery scales on the skin. It is difficult to cure and prone to relapse. The psoriasis commonly occurs on scalp and joints of limbs of a patient, it can spread to an entire body in severe cases. This disease is stubborn and difficult to treat, and it is often accompanied by complications such as hypertension, diabetes, and cardiovascular diseases. Severe psoriasis not only affects skin appearance and social activities of the patient, but also significantly impacts quality of life of the patient, causing great physical and psychological harm. In addition, improper treatment can even severely endanger patient's life. The psoriasis can be induced by a variety of factors, with a relatively high incidence and disability rate. At present, for patients with mild psoriasis, treatment options include topical corticosteroids, vitamin D derivatives, calcineurin inhibitors, keratolytic agents, and targeted phototherapy. For patients with the severe psoriasis, four classes of biologics are used for treatment: tumor necrosis factor (TNF) inhibitors, interleukin-12 and interleukin-23 (IL-12/23) inhibitors, interleukin-17 (IL-17) inhibitors, and interleukin-23 (IL-23) inhibitors. Traditional therapies are mostly expensive, with numerous side effects, and there is an urgent need to develop a safe, effective, inexpensive, and readily available small-molecule compound.

Inflammatory bowel disease (IBD) is a series of diseases including ulcerative colitis (UC) and Crohn's disease (CD). These diseases are characterized by chronic intestinal inflammation, which often leads to mucosal ulcers and progressive loss of intestinal function. At present, due to complex etiology and pathophysiology of the IBD, the IBD involve multiple factors such as genetics, environments, epithelial cells, microbiota, and immune responses, there is no effective treatment method.

Styrene compounds are a class of compounds with a structural framework where an ethylene double bond is connected to a benzene ring. Current literature reports indicate that styrene frameworks hold significant value in biomedical applications. In 2023, Food and drug administration (FDA) approved a VTAMA® (tapinarof, 1%) cream for a topical treatment of adult plaque psoriasis. This is a first non-steroidal topical drug and also a first-in-class topical new molecular entity drug approved for psoriasis treatment in the United States (US) in over 25 years.

### SUMMARY

To solve problems existing in the related art, a purpose of the disclosure is to provide a stilbene derivative, a preparation method thereof and an application thereof in preparation of drugs for treating psoriasis or ulcerative colitis. The disclosure provides a compound with a styrene structural framework, which exhibits excellent inhibitory activity against inflammatory skin diseases, particularly psoriasis. Compared to marketed drugs, The compound provided by the disclosure takes effect more quickly, has better anti-psoriasis efficacy, and demonstrates lower toxicity.

To achieve the above purpose, the disclosure provides the following solutions.

In a first aspect, the disclosure provides a stilbene derivative, including a compound II-2 and a compound II-23, and structural formulas of the compound II-2 and the compound II-23 are respectively as follows:

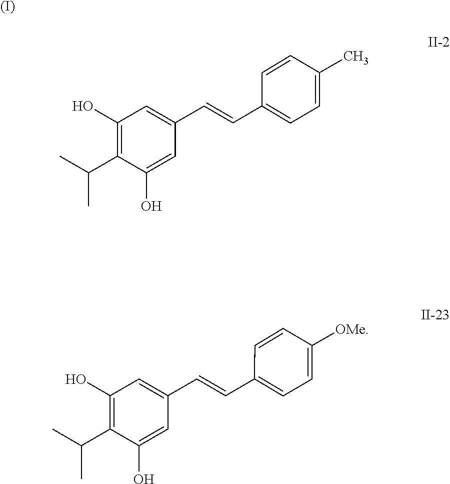

In a second aspect, the disclosure provides a preparation method of the stilbene derivative, including the following steps:
step (1), dissolving methyl 3,5-dimethoxybenzoate and 2-bromopropane in 1,2-dichloroethane, followed by adding aluminum chloride, and carrying out a substitution reaction to obtain a first intermediate;
step (2), adding boron tribromide to the first intermediate, and carrying a substitution reaction to obtain a second intermediate;
step (3), adding N,N-diisopropylethylamine and bromomethyl methyl ether to the second intermediate, and carrying out a substitution reaction to obtain a third intermediate;
step (4), adding lithium aluminum hydride to the third intermediate, and carrying out a reaction to obtain a fourth intermediate;
step (5), adding potassium carbonate and pyridinium chlorochromate to the fourth intermediate, and carrying out an oxidation reaction to obtain a fifth intermediate;
step (6), adding sodium hydride and a phosphate ester to the fifth intermediate, and carrying out a substitution reaction to obtain a sixth intermediate or a seventh intermediate; and
step (7), adding concentrated hydrochloric acid to the sixth intermediate or the seventh intermediate, and carrying out a substitution reaction to obtain the compound II-2 or the compound II-23.

In a third aspect, the disclosure provides an application of the stilbene derivative in preparation of drugs for treating psoriasis or ulcerative colitis.

Based on the above technical solutions, technical effects achieved by embodiments of the disclosure may be as follows.
(1) Stilbene framework derivatives II-2 and II-23 (i.e., the compound II-2 and the compound II-23) provided by the disclosure can significantly inhibit occurrence of psoriasis symptoms. In addition, the compound II-23 can alleviate symptoms of ulcerative colitis and has a therapeutic effect on ulcerative colitis in mice to a certain extent.
(2) The stilbene framework derivatives II-2 and II-23 provided by the disclosure can be obtained from the methyl 3,5-dimethoxybenzoate as a starting material through a sequence of reactions. The reactions include a Friedel-Crafts alkylation reaction, demethylation protection, reprotection with methoxymethyl, lithium aluminum hydride reduction, pyridinium chlorochromate (PCC) oxidation, a Horner-Wadsworth-Emmons reaction, and a re-deprotection reaction. A final yield of products is approximately 23%.

### BRIEF DESCRIPTION OF DRAWINGS

In order to more clearly describe technical solutions in embodiments of the disclosure or in the related art, a brief introduction to the drawings used in the embodiments is provided below. It is apparent that the drawings described below are merely some embodiments of the disclosure. For those skilled in the art, other drawings can also be obtained based on these drawings without involving any inventive step.

In FIG. 1, A and B illustrate that the compound II-2 and the compound II-23 can activate aryl hydrocarbon receptor (AHR) and promote its translocation to a nucleus. C illustrates that the compound II-2 and the compound II-23 can activate an AHR downstream pathway, thereby promoting expression of cytochrome P450 family 1 subfamily A member 1 (CYP1A1) and cytochrome P450 family 1 subfamily B member 1 (CYP1B1). An effect of the compound II-2 is comparable to that of a positive control drug, and the compound II-23 shows a superior effect. D illustrates that the compound II-2 and the compound II-23 can reduce expression of C—C motif chemokine ligand 5 (CCL5), C—C motif chemokine ligand 20 (CCL20), interleukin-6 (IL-6), interleukin-8 (IL-8), 5100 calcium binding protein A9 (S100A9), toll-like receptor 4 (TLR4), tumor necrosis factor-alpha (TNF-α), and tumor necrosis factor receptor 1 (TNFR1) in human cultured keratinocyte (HaCaT) cells.

FIG. 2A illustrates a principal component analysis (PCA) that demonstrates homogeneity of grouping. FIG. 2B illustrates numbers of differentially expressed genes between different groups. FIG. 2C and FIG. 2D illustrate volcano plots of the differentially expressed genes between the different groups. FIG. 2E illustrates a gene ontology (GO) enrichment plot. FIG. 2F illustrates a Kyoto encyclopedia of genes and genomes (KEGG) enrichment plot. FIG. 2G illustrates a heatmap of the differentially expressed genes with activation of the AHR downstream pathway.

FIG. 3 illustrates that with increase of administration days, imiquimod (IMQ) can gradually induce psoriasis symptoms in skin surfaces of mice, which are manifested as redness, scaling, and thickening. The compound II-2 and the compound II-23 can significantly inhibit occurrence of the psoriasis symptoms.

FIGS. 4A-4H illustrate that, as indicated by body weight change, body weight of a model group gradually decreased, and the compound II-2 and the compound II-23 are able to maintain increase in the body weight of the mice. From scoring results across multiple dimensions, the compound II-2 and the compound II-23 can inhibit the occurrence and development of the psoriasis symptoms on backs of the mice induced by the IMQ. The model group begins to show mild psoriasis symptoms on the second day of administration, which gradually worsened. The compound II-2 and the compound II-23 begin to show mild symptoms on the third day and are able to inhibit worsening of the symptoms. Compared with a normal group, IMQ can induce changes in messenger ribonucleic acid (mRNA) levels of inflammatory factors in skin tissues of psoriasis mice. Different concentrations of the compound II-2 and the compound II-23 show certain improvements. Specifically, FIG. 4A illustrates the body weight change of different groups. FIG. 4B illustrates psoriasis area and severity index (PASI) scores of the different groups. FIG. 4C illustrates skin thickness scores of the different groups. FIG. 4D illustrates total scores of the different groups. FIG. 4E illustrates effects of the compound II-2 and the compound II-23 on mRNA levels of cluster of differentiation 206 (CD206). FIG. 4F illustrates effects of the compound II-2 and the compound II-23 on mRNA levels of cluster of differentiation 36 (CD36). FIG. 4G illustrates effects of the compound II-2 and the compound II-23 on mRNA levels of interleukin-18 (IL-18). FIG. 4H illustrates effects of the compound II-2 and the compound II-23 on mRNA levels of monocyte chemoattractant protein-1 (MCP-1).

FIGS. 5A-5E illustrate that, as indicated by colonic length, degree of body weight change, and disease activity index (DAI) score in the mice, the compound II-23 can improve dextran sulfate sodium (DSS)-induced colon shortening and body weight loss in the mice, and also improve an overall condition of rectal bleeding in the mice. Hematoxylin and eosin (HE) staining results illustrate that the compound II-23 can mitigate DSS-induced colonic ulcers and, to some extent, suppress symptoms of ulcerative colitis in the mice, indicating its therapeutic potential for the ulcerative colitis. Specifically, FIG. 5A illustrates a gross appearance of the colon. FIG. 5B illustrates colonic length records of different groups. FIG. 5C illustrates HE staining of the colon of the different groups. FIG. 5D illustrates body weight records of the different groups. FIG. 5E illustrates DAI scores of the different groups.

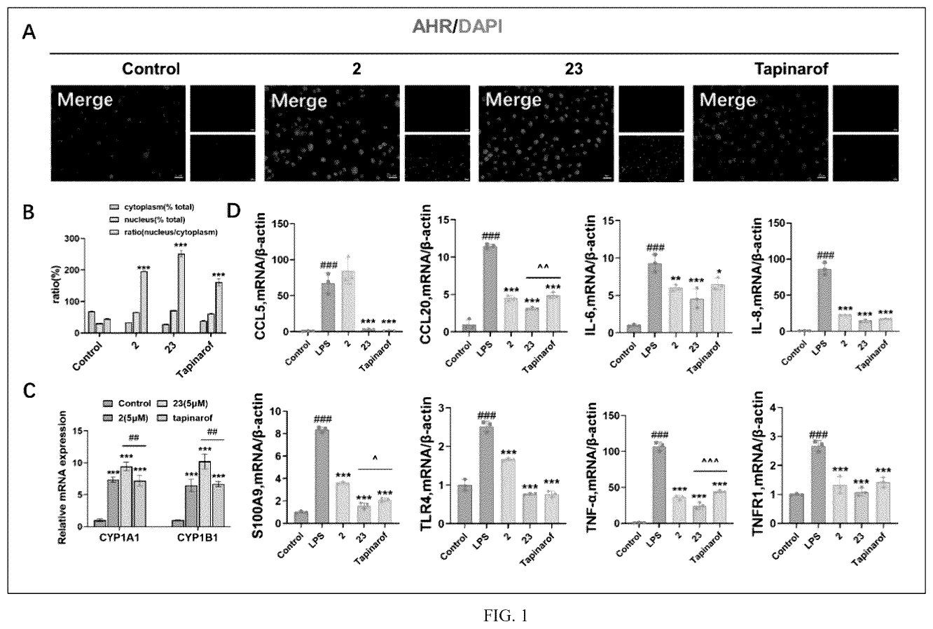

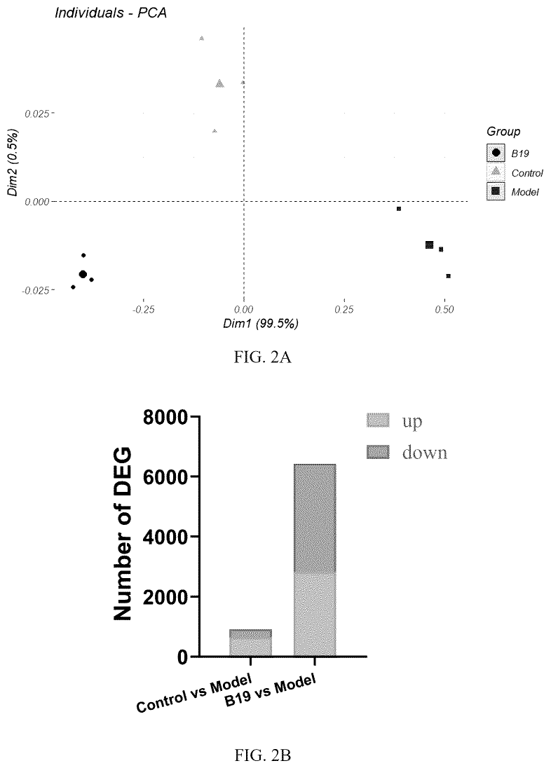

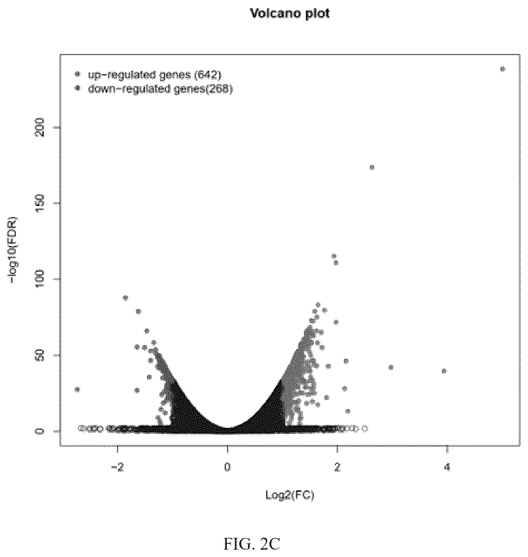

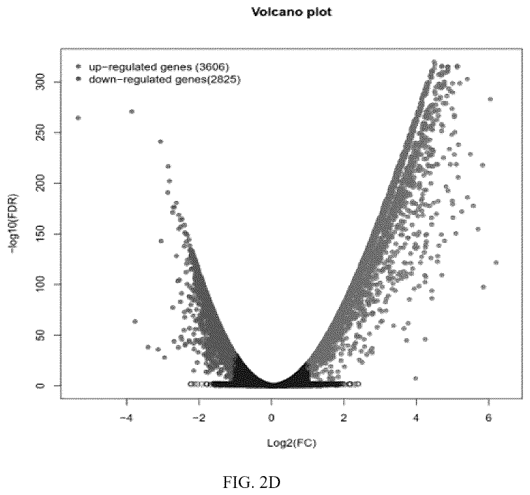

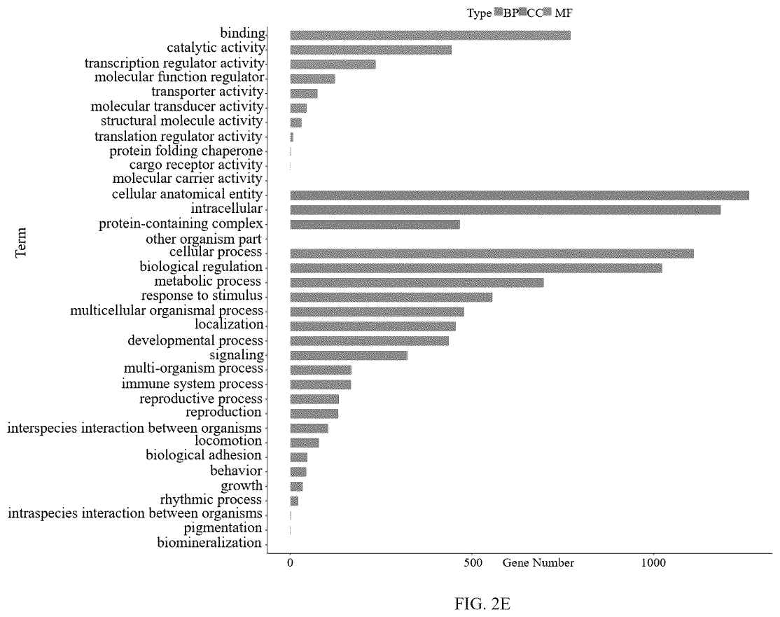

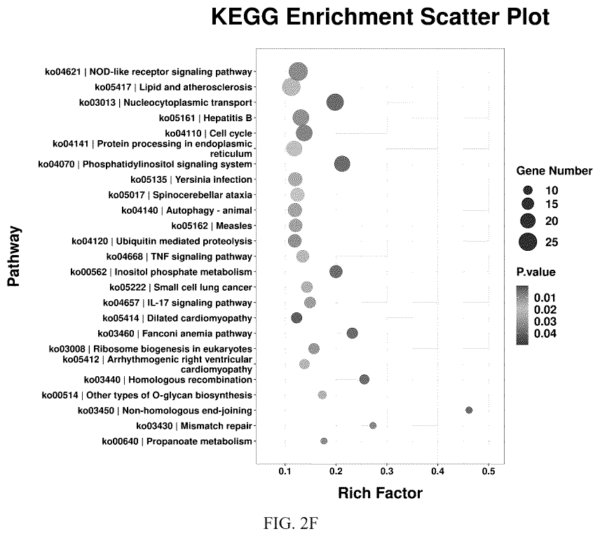

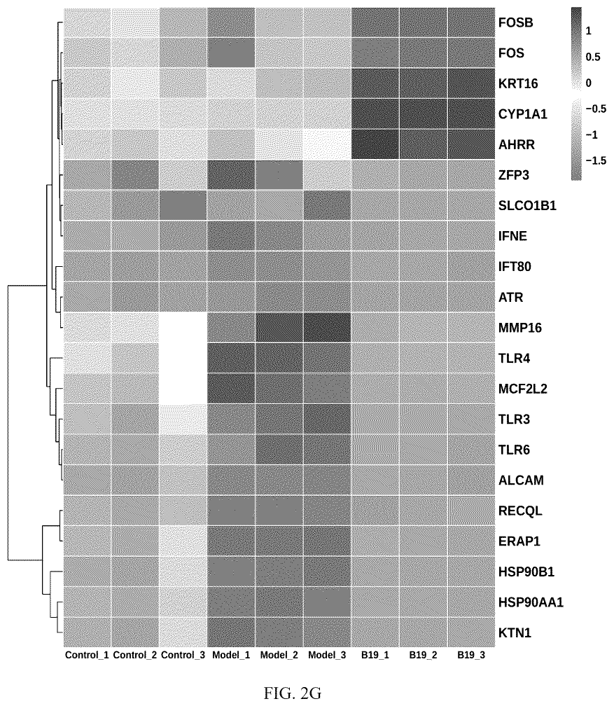

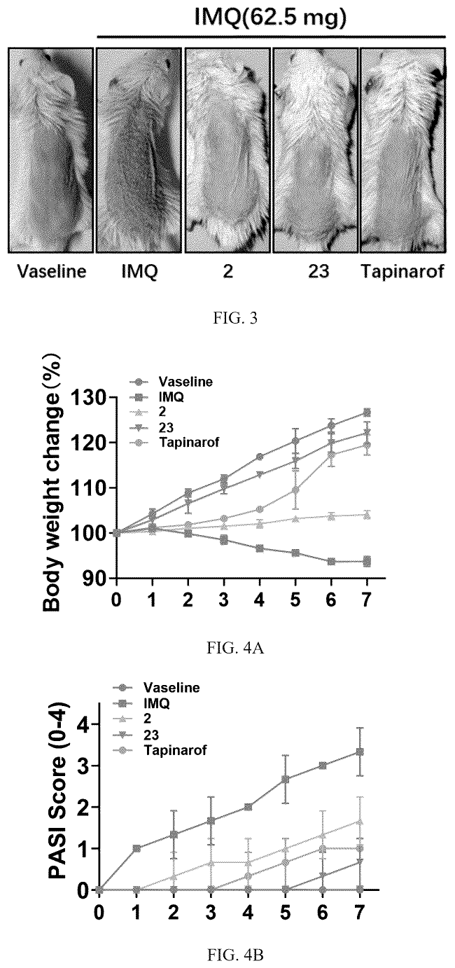

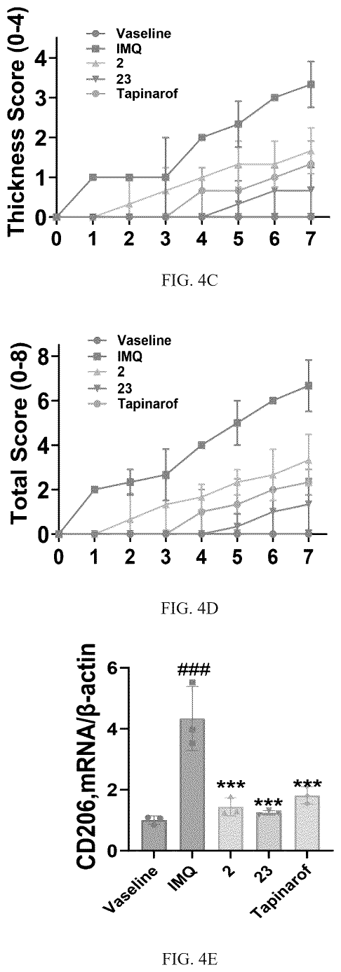

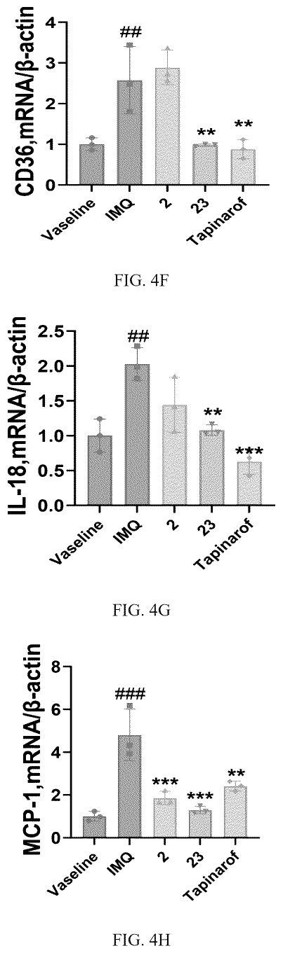

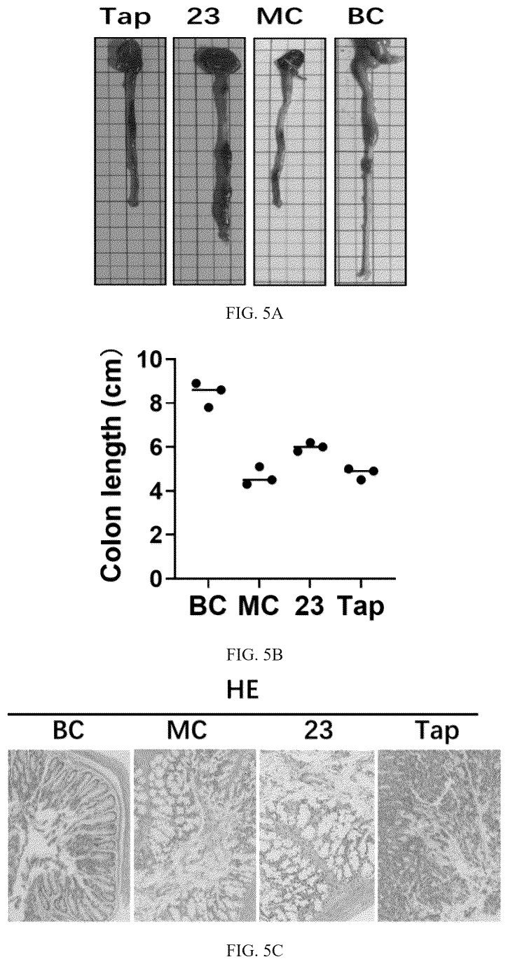

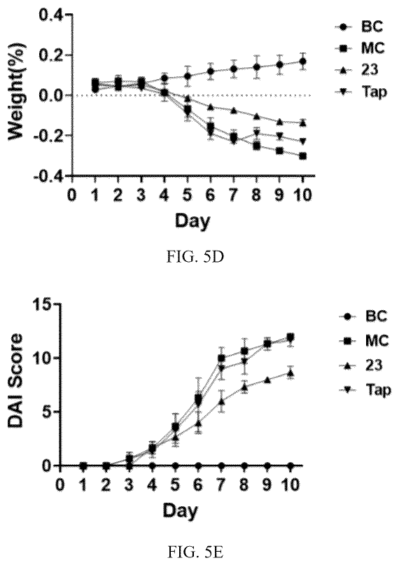

### DETAILED DESCRIPTION OF EMBODIMENTS

The disclosure is now described in detail with reference to various illustrative embodiments. This detailed description should not be construed as limiting the disclosure, but should be understood as a more detailed description of certain aspects, features, and embodiments of the disclosure.

It should be understood that the terms used in the disclosure are only for describing specific embodiments and are not intended to limit the disclosure. In addition, for a numerical range in the disclosure, it should be understood that each intermediate value between the upper and lower limits of the range is also specifically disclosed. Any intermediate value within any stated value or range, as well as any smaller range between any other stated value or intermediate value within the range, are also included in the disclosure. These smaller upper and lower limits can be independently included or excluded within the range.

Unless otherwise specified, all technical and scientific terms used herein have the same meaning as commonly understood by those skilled in the art to which the disclosure belongs. Although only specific methods and materials are described in the disclosure, any methods and materials similar or equivalent to those described herein may also be used in the practice or testing of the disclosure. All publications mentioned in this specification are incorporated by reference to disclose and describe methods and/or materials related to the cited publications. In the event of a conflict between any incorporated publications and the content of this specification, the content of this specification shall prevail.

Without departing from the scope or spirit of the disclosure, various modifications and changes can be made to the specific embodiments described in the specification of the disclosure, which would be obvious to those skilled in the art. Other embodiments that can be derived from the specification of the disclosure would also be obvious to those skilled in the art. The specification and embodiments of the disclosure are merely illustrative.

The terms “comprising”, “including”, “having”, “containing” and the like used in this text are open-ended terms, meaning they include but are not limited to.

The technical solutions described in the disclosure, unless specifically stated otherwise, are conventional solutions in the field. The reagents or materials used, unless specifically stated otherwise, are commercially available or publicly disclosed.

An embodiment of the disclosure provides a stilbene derivative, which includes a compound II-2 and a compound II-23. Structural formulas of the compound II-2 and the compound II-23 are respectively as follows:

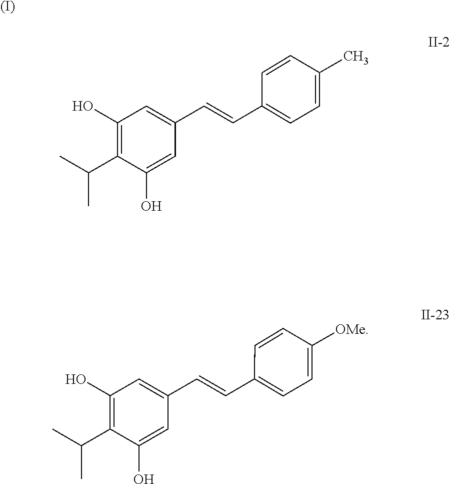

Another embodiment of the disclosure provides a preparation method of the stilbene derivative, which includes the following steps.

Step (1), methyl 3,5-dimethoxybenzoate and 2-bromopropane are dissolved in 1,2-dichloroethane, then anhydrous aluminum chloride is added, and a substitution reaction is carried out to obtain a first intermediate.

Step (2), boron tribromide is added to the first intermediate, and a substitution reaction is carried out to obtain a second intermediate.

Step (3), N,N-diisopropylethylamine and bromomethyl methyl ether are added to the second intermediate, and a substitution reaction is carried out to obtain a third intermediate.

Step (4), lithium aluminum hydride is added to the third intermediate, and a reaction is carried out to obtain a fourth intermediate.

Step (5), potassium carbonate and pyridinium chlorochromate are added to the fourth intermediate, and an oxidation reaction is carried out to obtain a fifth intermediate.

Step (6), sodium hydride and a phosphate ester are added to the fifth intermediate, and a substitution reaction is carried out to obtain a sixth intermediate or a seventh intermediate.

Step (7), concentrated hydrochloric acid is added to the sixth intermediate or the seventh intermediate, and a substitution reaction is carried out to obtain the compound II-2 and the compound II-23.

In some specific embodiments, in the step (1), a molar ratio of the 2-bromopropane to the methyl 3,5-dimethoxybenzoate is (1.1-1.3):1. A molar ratio of the aluminum chloride to the methyl 3,5-dimethoxybenzoate is (1.05-1.2):1. The substitution reaction is carried out at 85° C. to 95° C. for 5 hours to 8 hours.

In some specific embodiments, in the step (2), a molar ratio of the boron tribromide to the first intermediate is (4.0-7.0):1, and the substitution reaction is carried out under an inert atmosphere at 25° C. to 35° C. for 10 hours to 15 hours.

In some specific embodiments, in the step (3), a molar ratio of the N,N-diisopropylethylamine, the bromomethyl methyl ether, and the second intermediate is (10-15):(10-15):1, and the substitution reaction is carried out at 25° C. to 35° C. for 10 hours to 15 hours.

In some specific embodiments, in the step (4), a molar ratio of the third intermediate to the lithium aluminum hydride is (1-3):1, and the reaction is carried out under an inert atmosphere at 0° C. for 30 minutes.

In some specific embodiments, in the step (5), a molar ratio of the potassium carbonate, the pyridinium chlorochromate, and the fourth intermediate is (0.5-0.7):(2-2.5):1, and the oxidation reaction is carried out at 25° C. to 35° C. for 2 hours to 4 hours.

In some specific embodiments, in the step (6), a molar ratio of the sodium hydride, the phosphate ester, and the fifth intermediate is (1.2-1.5):(1.2-1.5):1, and the substitution reaction is carried out at 25° C. to 35° C. for 6 hours to 10 hours. The phosphate ester is diethyl (4-methoxybenzyl)phosphonate or diethyl (4-methylbenzyl)phosphonate.

In some specific embodiments, in the step (7), a molar ratio of the concentrated hydrochloric acid to the sixth intermediate or the seventh intermediate is 1 milliliter (mL):(1-1.2) mole (mol), and the substitution reaction is carried out at 25° C. to 35° C. for 2 hours to 4 hours.

Still another embodiment of the disclosure provides an application of the stilbene derivative in preparation of drugs for treating psoriasis or ulcerative colitis.

The compound II-2 and the compound II-23 provided by the disclosure target activation of AHR related pathways, promoting translocation of AHR to a nucleus, and reducing levels of related cytokines at a cellular level. The compound II-2 and the compound II-23 can significantly improve skin symptoms and PASI scores in an IMQ-induced psoriasis mouse model. Compared to lead compound tapinarof, the compound II-2 and the compound II-23 exhibit superior anti-psoriasis activity and lower toxicity. In addition, the compound II-23 can effectively treat ulcerative colitis in mice, reducing inflammatory responses and demonstrating lower toxicity. This represents an expanded therapeutic indication not present in the lead compound tapinarof. The compound II-2 and the compound II-23 of the disclosure can be used to prepare drugs for treating psoriasis, atopic dermatitis, and other skin diseases, as well as ulcerative colitis.

### First Embodiment

A stilbene derivative, a preparation process of which is as follows:

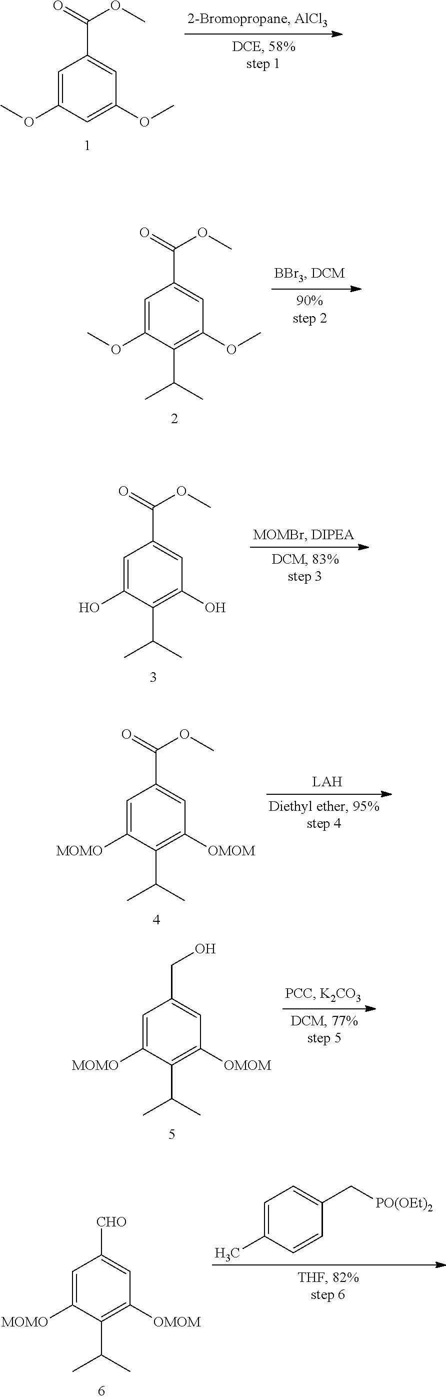

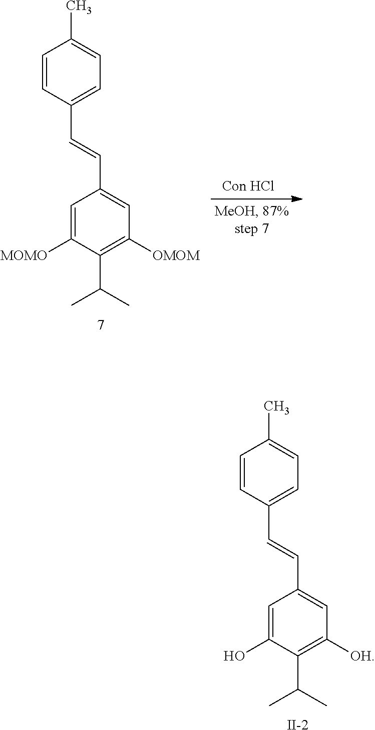

A preparation method of the stilbene derivative includes the following steps.

Step (1), methyl 3,5-dimethoxybenzoate (2 millimole (mmol), 1.0 equivalent) is dissolved in 10 mL of 1,2-dichloroethane (DCE, also referred to as EDC). At room temperature, 2-bromopropane (2.4 mmol, 1.2 equivalent) and aluminum chloride (AlCl₃, 2.4 mmol, 1.2 equivalent) are added in sequence to obtain a mixture. The mixture is transferred to an oil bath at 90° C. and reacted for 6 hours to obtain a reacted mixture. Then the reacted mixture is cooled to the room temperature, and 30 mL of saturated sodium bicarbonate solution is added to the reacted mixture to quench the reaction, thereby to obtain a solution with producing a flocculent precipitate. The solution is clarified by adding 30 mL of dilute hydrochloric acid, followed by adding ethyl acetate to extract organic phases three times. The organic phases are combined to obtain a combined organic phase. The combined organic phase is washed three times with saturated sodium chloride, dried with anhydrous sodium sulfate, concentrated by rotary evaporation, and purified by column chromatography (petroleum ether (PE) ethyl acetate (EA)=200:1) to obtain a first intermediate with a yield of 58%.

Step (2), in a reaction vessel, the first intermediate (1.0 equivalent) is dissolved in anhydrous dichloromethane (DCM), and air of the reaction vessel is purged with argon three times. Then boron tribromide (BBr₃, 1 mol per liter (M) in DCM, 5 equivalent) is added dropwise at −78° C. to obtain a mixture. After then, the mixture is gradually warmed to the room temperature and reacted overnight to obtain a reacted mixture. A temperature of the reacted mixture is then reduced to −78° C. Then anhydrous methanol is added to the reacted mixture to quench the reaction, followed by purification using column chromatography (PE:EA=5:1) to obtain a second intermediate with a yield of 90%.

Step (3), in a reaction vessel, the second intermediate (1.0 equivalent) is dissolved in anhydrous DCM, and air of the reaction vessel is purged with argon three times. At 0° C., N,N-diisopropylethylamine (DIPEA, 15 equivalent) is added, followed by a slow dropwise addition of bromomethyl methyl ether (MOMBr, 15 equivalent) to obtain a mixture. Areaction is carried out on the mixture for 1 hour, then a temperature is gradually raised to the room temperature, and the reaction is continued for 10 hours to obtain a reacted mixture. After monitoring completion of the reaction by thin layer chromatography (TLC), the reacted mixture is washed twice with saturated sodium bicarbonate, washed twice with saturated sodium chloride, dried with anhydrous sodium sulfate, concentrated by rotary evaporation, and purified by column chromatography (PE:EA=20:1) to obtain a third intermediate with a yield of 83%.

Step (4), in a reaction vessel, the third intermediate (1.0 equivalent) is dissolved in anhydrous diethyl ether, and air of the reaction vessel is purged with argon three times. At 0° C., lithium aluminum hydride (LAH, 2.5 mol/L in tetrahydrofuran (THF), 0.5 equivalent) is added dropwise, followed by reacting at 0° C. for 30 minutes to obtain a reacted mixture. Then 1.0 equivalent of water, 1.0 equivalent of 15% sodium hydroxide solution, and 3.0 equivalent of water are added sequentially to the reacted mixture to obtain a mixed solution. The mixed solution is then transferred to the room temperature and stirred for an additional 15 minutes, followed by filtration through diatomaceous earth to obtain a filtrate. The filtrate is washed twice with saturated sodium chloride, dried with anhydrous sodium sulfate, and concentrated by filtration and rotary evaporation to obtain a fourth intermediate with a yield of 95%.

Step (5), the fourth intermediate (1.0 equivalent) is dissolved in anhydrous DCM. At 0° C., potassium carbonate (K₂CO₃, 0.5 equivalent) and pyridinium chlorochromate (PCC, 2.2 equivalent) are added in sequence to obtain a mixture. A reaction is then carried out on the mixture at the room temperature for 2.5 hours to obtain a reacted mixture. Then ether is added to the reacted mixture to quench the reaction, followed by filtration through diatomaceous earth to obtain a filtrate. The filtrate is concentrated by rotary evaporation, and purified by column chromatography (PE:EA=20:1) to obtain a fifth intermediate with a yield of 77%.

Step (6), in a reaction vessel, diethyl (4-methylbenzyl)phosphonate (1.2 equivalent) is dissolved in THF, and air of the reaction vessel is purged with argon three times. Then sodium hydride (60% in mineral oil, 1.5 equivalent) is added at 0° C. to obtain a mixture. The mixture is reacted for 30 minutes, followed by adding the fifth intermediate (1.0 equivalent) to obtain a reacted mixture. The reacted mixture is transferred to the room temperature and reacted for 2 hours to obtain a final reacted mixture. Then water is added to the final reacted mixture at 0° C. to quenched the reaction, followed by adding ethyl acetate to extract organic phases three times. The organic phases are combined to obtain a combined organic phase. The combined organic phase is washed three times with saturated sodium chloride solution, dried with anhydrous sodium sulfate, concentrated by rotary evaporation, and purified by column chromatography (PE:EA=20:1) to obtain a sixth intermediate with a yield of 82%.

Step (7), the sixth intermediate is dissolved in 10 mL of anhydrous methanol, followed by adding 1 mL of concentrated hydrochloric acid (Con HCl) to obtain a mixture. A reaction is then carried out on the mixture at the room temperature for 2 hours to obtain a reacted mixture. The reacted mixture is concentrated by rotary evaporation, and purified by column chromatography (PE:EA=10:1) to obtain a compound 11-2 with a yield of 87%.

The compound 11-2: ¹H NMR (600 megahertz abbreviated as MHz, Chloroform-d) δ 7.36 (d, J=8.0 hertz abbreviated as Hz, 2H), 7.15 (d, J=7.9 Hz, 2H), 6.95 (d, J=16.2 Hz, 1H), 6.85 (d, J=16.3 Hz, 1H), 6.48 (s, 2H), 4.80 (s, 2H), 3.44 (hept, J=7.1 Hz, 1H), 2.35 (s, 3H), 1.38 (s, 3H), 1.36 (s, 3H).

### Second Embodiment

A stilbene derivative, a preparation process of which is as follows:

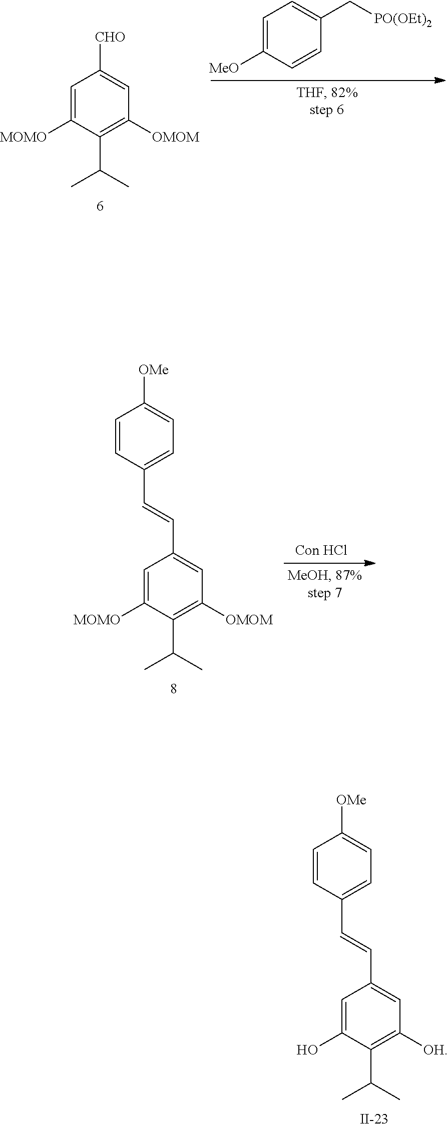

Step (1) to step (5) are the same as those in the first embodiment.

Step (6), in a reaction vessel, diethyl (4-methoxybenzyl)phosphonate (1.2 equivalent) is dissolved in THF, and air of the reaction vessel is purged with argon three times. Then sodium hydride (60% in mineral oil, 1.5 equivalent) is added at 0° C. to obtain a mixture. The mixture is reacted for 30 minutes, followed by adding the fifth intermediate (1.0 equivalent) to obtain a reacted mixture. The reacted mixture is transferred to the room temperature and reacted for 2 hours to obtain a final reacted mixture. Then water is added to the final reacted mixture at 0° C. to quench the reaction, followed by adding ethyl acetate to extract organic phases three times. The organic phases are combined to obtain a combined organic phase. The combined organic phase is washed three times with saturated sodium chloride solution, dried with anhydrous sodium sulfate, concentrated by rotary evaporation, and purified by column chromatography (PE:EA=20:1) to obtain a seventh intermediate with a yield of 82%

Step (7), the seventh intermediate is dissolved in 10 mL of anhydrous methanol, followed by adding 1 mL of concentrated hydrochloric acid to obtain a mixture. A reaction is then carried out on the mixture at the room temperature for 2 hours to obtain a reacted mixture. The reacted mixture is concentrated y rotary evaporation, and purified by column chromatography (PE:EA=10:1) to obtain a compound II-23 with a yield of 87%.

The compound II-23: ¹H NMR (400 MHz, CDCl₃) δ 7.39 (d, J=8.6 Hz, 2H), 6.91 (d, J=16.2 Hz, 1H), 6.87 (d, J=8.6 Hz, 2H), 6.75 (d, J=16.3 Hz, 1H), 6.45 (s, 2H), 4.78 (s, 2H), 3.80 (s, 3H), 3.42 (p, J=7.1 Hz, 1H), 1.36 (s, 3H), 1.34 (s, 3H).

### Third Embodiment

Transcription levels of AHR in HaCaT cells transfected with knockout lentivirus are measured before and after treatment with the compound II-2, the compound II-23, and tapinarof to evaluate their AHR agonist activity. Results are shown in Table 1.

TABLE 1
AHR agonist activity

| compound | II-2 | II-23 | tapinarof |
|---|---|---|---|
| half-maximal effective concentration | 8.41 | 2.01 | 28.93 |
| (EC₅₀) (nanomoles per liter | | | |
| abbreviated as nM) | | | |

It can be seen from data in Table 1, EC₅₀ values of the compound II-2 and the compound II-23 provided by the disclosure are significantly lower than a EC₅₀ value of a positive control drug (i.e., the tapinarof), indicating that the compound II-2 and the compound II-23 have stronger AHR agonist activity.

### Fourth Embodiment

1. Sterile Cell Culture Slides of Appropriate Size are Placed into a Six-Well Cell Culture Plate (1 Insert Per Well).

HaCaT cells in good growth condition are collected and diluted to a HaCaT cell suspension with a density of 1×10⁶ cells per milliliter. Then the HaCaT cell suspension is seeded into the six-well cell culture plate and cultured in an incubator with a culture medium for 24 hours, followed by treating a compound. After 24 hours of treatment, supernatant is removed, and the culture medium is discarded. HaCaT cells after treatment are washed three times with phosphate buffered saline (PBS), followed by fixing with 4% paraformaldehyde for 20 minutes and washing three times with PBS. A histology pen is used to draw a circle around HaCaT cell culture slides to prevent a subsequent incubation solution from flowing away. Then the HaCaT cell culture slides are washed with PBS, followed by blocking with 5% bovine serum albumin (BSA) (Biofroxx, 4240GR250) for 2 hours, adding an appropriate amount of primary antibody (AHR, 1:1000, Cell Signaling Technology, USA) working solution, and incubating overnight at 4° C. After warming to room temperature, the HaCaT cell culture slides are washed three times with PBS for 5 minutes each time. A corresponding secondary antibody (FITC-conjugated anti-rabbit) working solution is added within the circle, and the HaCaT cell culture slides are incubated in a water bath at 37° C. in a dark environment for 40 minutes, followed by washing three times with PBS for 5 minutes each time. 4′,6-diamidino-2-phenylindole (DAPI) (Sigma, D8417-1MG) is added to stain nuclei, and HaCaT cell culture slides are incubated in the dark environment at room temperature for 20 to 30 minutes, followed by washing with PBS. The HaCaT cell culture slides are mounted with an anti-fade mounting medium (Sigma, V900155-25G) and stored in a dark box at 4° C. Then the HaCaT cell culture slides are then observed and photographed under a microscope (OLYMPUS, IX51).

An experimental result illustrates that after drug administration, green fluorescence of AHR migrates to a nucleus, indicating that the compound activates AHR to a certain extent and promotes its translocation into the nucleus.

2. Detecting Changes in mRNA Levels of Inflammatory Factors in Cells and Skin Tissues by Quantitative Real-Time Polymerase Chain Reaction (qRT-PCR)
(1) RNA extraction from the cells: HaCaT cells are evenly seeded in a six-well plate and cultured according to experimental groups. HaCaT cells from different groups are then collected into 1.5 mL centrifuge tubes and gently washed 2-3 times with pre-cooled PBS. After adding 1 mL of Trizol reagent and incubating for 5 minutes, 0.2 mL of chloroform is added to each of the centrifuge tubes, and each of the centrifuge tubes is gently inverted to mix. Each of the centrifuge tubes is then left undisturbed at room temperature for 10 minutes, followed by centrifugation at 10,000 times acceleration of gravity (10,000 g) for 15 minutes at 4° C. An upper aqueous phase of each of the centrifuge tubes is carefully transferred to another centrifuge tube, and isopropanol with a same volume as the upper aqueous phase is added to precipitate RNA. After gentle mixing and incubation at room temperature for 5 minutes, each of the centrifuge tubes is centrifuged for 10 minutes at 10,000 g. RNA precipitation at a bottom of each of the centrifuge tubes is retained, and washed with 1 mL of ethanol with a volume fraction 75% prepared with diethyl pyrocarbonate (DEPC) water to remove residual isopropanol. The ethanol solution is discarded after centrifugation. The RNA precipitation is then dried for 10 minutes at room temperature or under vacuum and dissolved in DEPC water (50 microliter (μL) per well) to obtain an RNA solution. An RNA concentration of the RNA solution is measured, and the RNA solution is stored at −80° C.
(2) RNA extraction from animal tissues: HaCaT cells are collected into 1.5 mL centrifuge tubes, and 1 mL of Trizol reagent and grinding beads are added. The centrifuge tubes are homogenized in a homogenizer. After homogenization, 0.2 mL of chloroform is added to each of the centrifuge tubes, and each of the centrifuge tubes is gently inverted to mix. Each of the centrifuge tubes is then incubated at room temperature for 10 minutes, followed by centrifugation at 4° C. and 10,000 g for 15 minutes. An upper aqueous phase of each of the centrifuge tubes is carefully transferred to another centrifuge tube, and isopropanol with a same volume as the upper aqueous phase is added to precipitate RNA. Each of the centrifuge tubes is gently mixed and incubated at room temperature for 5 minutes, then centrifuged for 10 minutes at 10,000 g. RNA precipitation at a bottom of each of the centrifuge tubes is retained, and washed with 1 mL of ethanol with a volume fraction 75% prepared with DEPC water to remove residual isopropanol. The ethanol solution is discarded after centrifugation. The RNA precipitation is then dried for 10 minutes at room temperature or under vacuum and dissolved in DEPC water (50 μL per well) to obtain an RNA solution. An RNA concentration of the RNA solution is measured, and the RNA solution is stored at −80° C.
(3) Reverse transcription: in a clean bench, an enzyme free Eppendorf (EP) tube is taken. 2 micrograms (g) of RNA and Hifair® III 1st Strand cDNA Synthesis SuperMix for qPCR (YEASON, Cat: 11141ES10) are added to the enzyme free EP tube to obtain a mixture. A total volume of the mixture is adjusted to 20 μL with DEPC water, followed by incubation in a heating block at 25° C. for 5 minutes, then at 42° C. for 40 minutes, and finally at 75° C. for 5 minutes.
(4) Polymerase chain reaction (PCR) amplification: a 10 μL reaction volume is prepared, which includes 2 μL of sample deoxyribonucleic acid (DNA), 2 μL of DEPC water, 1 μL of corresponding primers, and 5 μL of Hieff® qPCR SYBR Green Master Mix (YEASON, Cat: 11202ES01). Then qRT-PCR is performed using an ABI QuantStudio 5 PCR system (Thermo Fisher Scientific, USA). A temperature gradient is set as follows: 95° C. (5 minutes)→95° C. (10 seconds)→55-60° C. (20 seconds)→72° C. (30 seconds), and complementary DNA (cDNA) amplification is performed for 40 cycles.

Results show that after treatment with the compound II-2 and the compound II-23, expression of CCL5, CCL20, IL-6, IL-8, S100A9, TLR4, TNF-α, and TNFR1 in the cells is inhibited. The compound II-23 exhibits superior inhibitory activities against CCL20, S100A9, and TNF-α compared to tapinarof. These findings indicate that the compound II-2 and the compound II-23 can block inflammatory factors. Compared to IMQ treatment, the compound II-2 reduced levels of CD206 and MCP-1 in animal skin lesion tissues and demonstrated effects similar to tapinarof treatment. In addition to these factors, compared to the IMQ treatment, the compound II-23 also lowers levels of CD36 and IL-18.

### 3. Psoriasis Animal Modeling and Drug Administration Method

Eight-week-old male Balb/C mice are adaptively housed in a specific pathogen-free (SPF) grade barrier environment for one week. A central back hair of every mouse is removed using a mouse hair clipper. The mice are then randomly divided into five groups: a blank control group (Con), an IMQ model group (IMQ), a positive drug group (tapinarof), an II-2 group (1%), and an II-23 group (1%).

Mice in the IMQ model group, mice in the positive drug group, mice in the II-2 group, and mice in the II-23 group are treated with 62.5 mg of imiquimod for 7 days. During this period, the mice in the positive drug group are treated with tapinarof (1%), the mice in the II-2 group are treated with II-2 (1%), and the mice in the II-23 group are treated with II-23 (1%) via topical application. When the mice in the IMQ model group reach the criteria of weight loss, psoriasis-like skin lesions, and skin lesion proliferation, their PASI scores continue to rise. Therefore, a psoriasis-like mouse model is successfully established. Mice in the blank control group received no treatment. Dynamic changes of skin lesions on the back of the mice are observed and recorded daily before drug administration.

### PASI Scoring

The scoring is based on severity of psoriasis in the mice, specifically the degree of scaling, erythema, and induration of the skin lesions on the back. (0: no lesions; 1: mild lesions; 2: moderate lesions; 3: severe lesions; 4: very severe lesions). Three mice per group, with scores recorded once daily before drug administration.

Results show that the compound II-2 and the compound II-23 inhibits onset and exacerbation of IMQ-induced psoriasis symptoms on the back of the mice, which was mainly manifested as a reduction in the severity of erythema and scaling. Specifically, the compound II-23 does not exhibit these symptoms until the fifth day. The compound II-23 also demonstrates minimal toxicity, with no changes in body weight.

### 4. Ulcerative Colitis Animal Modeling and Drug Administration Method

After one week of adaptive housing in an SPF-grade barrier environment, eight-week-old male Balb/C mice are provided with free access to drinking water containing 2.8% DSS for one week. After three days of free drinking water, compound groups are treated with 20 milligrams per kilogram (mg/kg) of the compound II-23 or 20 mg/kg of tapinarof via oral gavage for seven consecutive days. Body weight of the mice is recorded, and a condition of rectal bleeding of the mice is observed. A blank control group is provided with free access to distilled water throughout the experiment and received carboxymethyl cellulose sodium via oral gavage. A DSS group is provided with free access to drinking the water containing 2.8% DSS for the first seven days and received carboxymethyl cellulose sodium via oral gavage. The compound II-23 group is provided with free access to drinking the water containing 2.8% DSS for the first seven days and received 20 mg/kg of the compound II-23 formulated in carboxymethyl cellulose sodium via oral gavage. The tapinarof group is provided with free access to drinking the water containing 2.8% DSS the first seven days and received 20 mg/kg of tapinarof formulated in carboxymethyl cellulose sodium via oral gavage.

### HE Staining Method for Detecting Pathological Changes in Colon Tissue

A portion of mouse colonic tissues is fixed in 4% paraformaldehyde, and embedded in paraffin and sectioned into slices. A specific operation is as follows. The slices are dehydrated from low to high concentration by immersing in xylene for 20 minutes. This process is repeated and the xylene is discarded. Then the slices are immersed in anhydrous ethanol for 5 minutes. This process is repeated and 75% ethanol is added to dewaxing for 5 minutes. Finally, the slices are washed with water to obtain rehydrated slices. Subsequently, hematoxylin staining is performed. The rehydrated slices are stained with hematoxylin for a few to several minutes, then acid-washed with a 1% hydrochloric acid solution to remove excess stain and differentiate, and finally treated with a basic solution to turn the nuclei blue. Next, eosin staining is performed. The slices, after hematoxylin staining, are stained with eosin for the cytoplasm, followed by dehydrating with graded ethanol (85% ethanol-95% ethanol) and then staining with eosin for a few minutes. Finally, dehydration and mounting for observation are carried out. The slices, after eosin staining, are immersed in absolute ethanol for several minutes, then placed in a clearing agent (usually xylene) for a few minutes, repeating this step once, and then air-dried. These slices are then photographed under a fluorescence microscope, with the nuclei appearing blue and the cytoplasm appearing red.

Results show that the colon length of the compound groups increases significantly compared to the DSS group, and body weight is restored. A HE staining result illustrates that the DSS group exhibits pronounced ulcers, and the compound groups alleviated these ulcers and achieved a certain level of therapeutic effect on ulcerative colitis. In contrast, the tapinarof group did not show significant improvements in body weight or colon length compared to the model group, and numerous ulcers are still present in the HE staining.

In summary, methyl 3,5-dimethoxybenzoate is used as a starting material in the disclosure. A group is introduced at a carbon-four position via a Friedel-Crafts alkylation reaction or a Suzuki reaction. Subsequently, methyl groups are removed, and hydroxyl groups are protected with methoxymethyl. The ester group is then reduced and oxidized to obtain a key intermediate aldehyde. The aldehyde reacts with diethyl phosphonate in a Horner-Wadsworth-Emmons reaction to produce an intermediate. Finally, the methoxymethyl protection is removed under acidic conditions to obtain final products, i.e., the compound II-2 and the compound II-23.

The compound II-2 and the compound II-23 can effectively inhibit occurrence and development of psoriasis and alleviate its symptoms. The activity of the compound II-2 and the compound II-23 is stronger than that of tapinarof. The compound II-23 also has a certain therapeutic effect on ulcerative colitis. The compound II-2 and the compound II-23 of the disclosure can be used to prepare drugs for treating psoriasis or ulcerative colitis.

Apparently, the above embodiments of the disclosure are merely examples provided to clearly illustrate the disclosure and are not intended to limit the scope of the disclosure. Based on the above description, those skilled in the art can make various other forms of modifications or changes. It is neither necessary nor possible to exhaustively enumerate all possible embodiments here. Any modifications, equivalent substitutions, and improvements made within the spirit and principles of the disclosure should be included within the scope of protection of the claims of the disclosure.

What is claimed is:

1. A stilbene derivative, comprising a compound II-2 and a compound II-23, wherein structural formulas of the compound II-2 and the compound II-23 are respectively as follows:

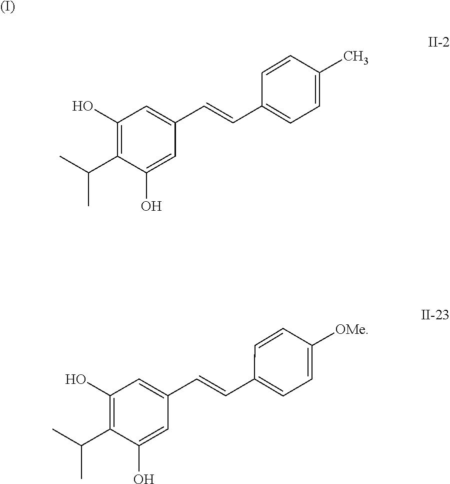

2. A preparation method of the stilbene derivative as claimed in claim 1, comprising the following steps:
step (1), dissolving methyl 3,5-dimethoxybenzoate and 2-bromopropane in 1,2-dichloroethane, followed by adding aluminum chloride, and carrying out a substitution reaction to obtain a first intermediate;
step (2), adding boron tribromide to the first intermediate, and carrying a substitution reaction to obtain a second intermediate;
step (3), adding N,N-diisopropylethylamine and bromomethyl methyl ether to the second intermediate, and carrying out a substitution reaction to obtain a third intermediate;
step (4), adding lithium aluminum hydride to the third intermediate, and carrying out a reaction to obtain a fourth intermediate;
step (5), adding potassium carbonate and pyridinium chlorochromate to the fourth intermediate, and carrying out an oxidation reaction to obtain a fifth intermediate;
step (6), adding sodium hydride and a phosphate ester to the fifth intermediate, and carrying out a substitution reaction to obtain a sixth intermediate or a seventh intermediate; and
step (7), adding concentrated hydrochloric acid to the sixth intermediate or the seventh intermediate, and carrying out a substitution reaction to obtain the compound II-2 or the compound II-23.

3. The preparation method as claimed in claim 2, wherein in the step (1), a molar ratio of the 2-bromopropane to the methyl 3,5-dimethoxybenzoate is (1.1-1.3):1, a molar ratio of the aluminum chloride to the methyl 3,5-dimethoxybenzoate is (1.05-1.2):1, and the substitution reaction is carried out at 85° C. to 95° C. for 5 hours to 8 hours.

4. The preparation method as claimed in claim 2, wherein in the step (2), a molar ratio of the boron tribromide to the first intermediate is (4.0-7.0):1, and the substitution reaction is carried out under an inert atmosphere at 25° C. to 35° C. for 10 hours to 15 hours.

5. The preparation method as claimed in claim 2, wherein in the step (3), a molar ratio of the N,N-diisopropylethylamine, the bromomethyl methyl ether, and the second intermediate is (10-15):(10-15):1, and the substitution reaction is carried out at 25° C. to 35° C. for 10 hours to 15 hours.

6. The preparation method as claimed in claim 2, wherein in the step (4), a molar ratio of the third intermediate to the lithium aluminum hydride is (1-3):1, and the reaction is carried out under an inert atmosphere at 0° C. for 30 minutes.

7. The preparation method as claimed in claim 2, wherein in the step (5), a molar ratio of the potassium carbonate, the pyridinium chlorochromate, and the fourth intermediate is (0.5-0.7):(2-2.5):1, and the oxidation reaction is carried out at 25° C. to 35° C. for 2 hours to 4 hours.

8. The preparation method as claimed in claim 2, wherein in the step (6), a molar ratio of the sodium hydride, the phosphate ester, and the fifth intermediate is (1.2-1.5):(1.2-1.5):1, the substitution reaction is carried out at 25° C. to 35° C. for 6 hours to 10 hours, and the phosphate ester is diethyl (4-methoxybenzyl)phosphonate or diethyl (4-methylbenzyl)phosphonate.

9. The preparation method as claimed in claim 2, wherein in the step (7), a molar ratio of the concentrated hydrochloric acid to the sixth intermediate or the seventh intermediate is 1 milliliter (mL):(1-1.2) mole (mol), and the substitution reaction is carried out at 25° C. to 35° C. for 2 hours to 4 hours.

10. An application method of the stilbene derivative as claimed in claim 1, comprising:
preparing drugs for treating psoriasis or ulcerative colitis by using the stilbene derivative.
